(19) United States
(12) Patent Application Publication
(10) Pub. No.: US 2012/0070885 A1
(43) Pub. Date: Mar. 22, 2012

(54) INTEGRATED DEVICE FOR ANALYZING AQUEOUS SAMPLES USING LIPID MULTILAYER GRATINGS

(75) Inventor: STEVEN LENHERT, Tallahassee, FL (US)

(73) Assignee: FLORIDA STATE UNIVERSITY RESEARCH FOUNDATION, TALLAHASSEE, FL (US)

(21) Appl. No.: 13/238,498

(22) Filed: Sep. 21, 2011

Related U.S. Application Data
(60) Provisional application No. 61/384,764, filed on Sep. 21, 2010.
(60) Provisional application No. 61/387,550, filed on Sep. 29, 2010.
(60) Provisional application No. 61/387,556, filed on Sep. 29, 2010.

(51) Int. Cl. C12M 1/34; G01N 21/00

(52) U.S. Cl. 435/288.7; 422/82.05

(57) ABSTRACT

Described is a device comprising lipid multilayer gratings.

### CROSS-REFERENCE TO RELATED APPLICATIONS

This application claims benefit of priority to: U.S. Provisional Patent Application No. 61/384,764 to Lenhert, entitled “NOVEL DEVICE FOR DETECTING AND ANALYZING AQUEOUS SAMPLES,” filed Sep. 21, 2010; U.S. Provisional Application No. 61/387,550 to Lenhert et al., entitled “LIPID MULTILAYER GRATINGS,” filed Sep. 29, 2010; and U.S. Provisional Application No. 61/387,556 to Lenhert, entitled “LIPID MULTILAYER GRATINGS FOR SEMI-SYNTHETIC QUORUM SENSORS,” filed Sep. 29, 2010, the entire content and disclosures of which are incorporated herein by reference in their entirety.

### BACKGROUND

1. Field of the Invention

The present invention relates to a device employing lipid multilayer gratings.

2. Related Art

Surface-supported phospholipid multilayers are promising materials for nanotechnology because of their tendency to self-organize, their innate biocompatibility, their ability to encapsulate other materials within the multilayers, and the ability to control the multilayer thickness between ˜2 and 100 nm during fabrication. Dip-pen nanolithography (DPN) is an atomic force microscopy (AFM) based fabrication method that allows high-throughput fabrication and integration of a variety of microstructured and nanostructured materials including lipid multilayers, with areal throughputs on the scale of cm² min¹. However, there are currently difficulties in trying to employ lipid multilayer gratings in analytical devices.

### SUMMARY

According to a broad aspect, the present invention provides a device comprising: a substrate, one or more sensors on the substrate, a reaction chamber containing the one or more sensors, an inlet to the reaction chamber for controlling inflow fluid into the reaction chamber, wherein each sensor of the plurality of sensors comprises a lipid multilayer grating.

### BRIEF DESCRIPTION OF THE DRAWINGS

The accompanying drawings, which are incorporated herein and constitute part of this specification, illustrate exemplary embodiments of the invention and, together with the general description given above and the detailed description given below, serve to explain the features of the invention.

FIG. 1 is a schematic drawing of a lab-on-a-chip sensor device according to one embodiment of the present invention.

FIG. 2 is a schematic drawing of a technique according to one embodiment of the present invention that may be used to fabricate lipid multilayer gratings.

FIG. 3 is an optical micrograph of light diffracted from gratings of different period that were fabricated in parallel with a one-dimensional tip array on a poly(methyl methacrylate) (PMMA) surface.

FIG. 4 is an AFM topographical image of a grating with a period of 600 nm and height of (29+3) nm.

FIG. 5 is a graph showing the correlation between the grating heights (measured by AFM) and the measured intensity of light diffracted from gratings with a period of 600 nm.

FIG. 6 is an optical micrograph of the diffraction from the gratings of FIG. 5 and their measured AFM heights.

FIG. 7 is a schematic drawing of waveguide grating couplers according to one embodiment of the present invention.

FIG. 8 is a photograph of a waveguide grating coupler according to one embodiment of the present invention at 30° from the surface normal.

FIG. 9 is a photograph of a waveguide grating coupler according to one embodiment of the present invention at 45° from the surface normal.

FIG. 10 is an image of red and green fluorescence for two different gratings according to one embodiment of the present invention.

FIG. 11 is an image showing two gratings where the individual elements within a single grating are composed of alternating materials.

FIG. 12 is a schematic diagram of three effects observed as a result of lipid adhesion with a substrate and interaction with protein from solution.

FIG. 13 is a fluorescence micrograph showing spreading of a lipid in air after minutes of exposure to humidity above 40%.

FIG. 14 is a fluorescence micrograph showing dewetting of smooth lines of biotin-containing gratings under solution to form droplets after 1 minute of exposure to the protein streptavidin.

FIG. 15 is a fluorescence micrograph showing intercalation of protein into lipid multilayer grating lines of different heights after 1 hour.

FIG. 16 shows the chemical structures of 1,2-dioleoyl-sn-glycero-3-phosphocholine (DOPC), a phospholipid, and 1,2-dioleoyl-sn-glycero-3-phosphoethanolamine-N-(lissamine rhodamine B sulfonyl) (DOPE-RB) used to make lipid multilayer gratings according to one embodiment of the present invention.

FIG. 17 is a graph showing label-free detection of protein binding by monitoring of the diffraction from gratings upon exposure to protein at different concentrations.

FIG. 18 is an optical micrograph of E. coli cells selectively adhered to a fluorescently labeled lipid microarray.

### DETAILED DESCRIPTION OF THE PREFERRED EMBODIMENTS

### Definitions

Where the definition of a term departs from the commonly used meaning of the term, applicant intends to utilize the definitions provided below, unless specifically indicated.

For purposes of the present invention, it should be noted that the singular forms, “a,” “an,” and “the” include reference to the plural unless the context as herein presented clearly indicates otherwise.

For purposes of the present invention, directional terms such as “top,” “bottom,” “upper,” “lower,” “above,” “below,” “left,” “right,” “horizontal,” “vertical,” “up,” “down,” etc. are merely used for convenience in describing the various embodiments of the present invention. The embodiments of the present invention may be oriented in various ways. For example, the diagrams, apparatuses, etc. shown in the drawing figures may be flipped over, rotated by 90° in any direction, reversed, etc.

For purposes of the present invention, a value or property is “based” on a particular value, property, the satisfaction of a condition, or other factor, if that value is derived by performing a mathematical calculation or logical decision using that value, property or other factor.

For purposes of the present invention, the term “analyte” refers to the conventional meaning of the term “analyte,” i.e., a substance or chemical constituent of a sample that is being detected or measured in a sample. In one embodiment of the present invention, the a sample to be analyzed may be an aqueous sample, but other types of samples may also be analyzed using a device of the present invention.

For purposes of the present invention, the term “array” refers to a one-dimensional or two-dimensional set of microstructures. An array may be any shape. For example, an array may be a series of microstructures arranged in a line, such as the array of squares. An array may be arranged in a square or rectangular grid. There may be sections of the array that are separated from other sections of the array by spaces. An array may have other shapes. For example, an array may be a series of microstructures arranged in a series of concentric circles, in a series of concentric squares, a series of concentric triangles, a series of curves, etc. The spacing between sections of an array or between microstructures in any array may be regular or may be different between particular sections or between particular pairs of microstructures. The microstructure arrays of the present invention may be comprised of microstructures having zero-dimensional, one-dimensional or two-dimensional shapes. The microstructures having two-dimensional shapes may have shapes such as squares, rectangles, circles, parallelograms, pentagons, hexagons, irregular shapes, etc.

For purposes of the present invention, the term “biomolecule” refers to the conventional meaning of the term biomolecule, i.e., a molecule produced by or found in living cells, e.g., a protein, a carbohydrate, a lipid, a phospholipid, a nucleic acid, etc.

For purposes of the present invention, the term “camera” refers to any type of camera or other device that senses light intensity. Examples of cameras include digital cameras, scanners, charged-coupled devices, CMOS sensors, photomultiplier tubes, analog cameras such as film cameras, etc. A camera may include additional lenses and filters such as the lenses of a microscope apparatus that may adjusted when the camera is calibrated.

For purposes of the present invention, the term “dehydrated lipid multilayer grating” refers to a lipid multilayer grating that is sufficiently low in water content that it is no longer in fluid phase.

For purposes of the present invention, the term “detector” refers to any type of device that detects or measures light. A camera is a type of detector.

For purposes of the present invention, the term “dot” refers to a microstructure that has a zero-dimensional shape.

For purposes of the present invention, the term “fluorescence” refers to the conventional meaning of the term fluorescence, i.e., the emission of light by a substance that has absorbed light or other electromagnetic radiation of a different wavelength.

For purposes of the present invention, the term “fluorescent” refers to any material or mixture of materials that exhibits fluorescence.

For purposes of the present invention, the term “fluorescent dye” refers to any substance or additive that is fluorescent or imparts fluorescence to another material. A fluorescent dye may be organic, inorganic, etc.

For purposes of the present invention, the term “fluorescent microstructure” refers to a microstructure that is fluorescent. A fluorescent microstructure may be made of a naturally fluorescent material or may be made of a nonfluorescent material, such as a phospholipid, doped with a fluorescent dye.

For purposes of the present invention, the term “fluorescent nanostructure” refers to a nanostructure that is fluorescent. A fluorescent nanostructure may be made of a naturally fluorescent material or may be made of a nonfluorescent material, such as a phospholipid, doped with a fluorescent dye.

For purposes of the present invention, the term “fluid” refers to a liquid or a gas.

For purposes of the present invention, the term “freezing by dehydration” refers to removal of residual water content, for instance by incubation in an atmosphere with low water content, for instance a vacuum (<50 mbar) or at relative humidity below 40% (at standard temperature and pressure).

For purposes of the present invention, the term “hardware and/or software” refers to functions that may be performed by digital software or digital hardware, or a combination of both digital hardware and digital software.

For purposes of the present invention, the term “grating” refers to an array of dots, lines, or a 2D shape that are regularly spaced at a distance that causes coherent scattering of incident light.

For purposes of the present invention, the term “inlet” refers to an apparatus for allowing the addition of a fluid, such as a liquid sample, at a controlled speed and direction to a reaction chamber or onto the sensors of a sensor device of the present invention.

For purposes of the present invention, the term “lab-on-a-chip” refers to a device that integrates one or several laboratory functions on a single chip of only millimeters to a few square centimeters in size. A lab-on-a-chip may be used in combination with a larger chip reader, e.g. including a camera.

For purposes of the present invention, the term “light,” unless specified otherwise, refers to any type of electromagnetic radiation. Although in the embodiments described below, the light that is incident on the gratings or sensors is visible light, the light that is incident on the gratings or sensor of the present invention may be any type of electromagnetic radiation including infrared light, ultraviolet light, etc. that may be scattered by a grating or sensor. Although in the embodiments described below, the light that is scattered from the gratings or sensors and detected by a detector is visible light, the light that is scattered by a grating or sensor of the present invention and detected by a detector of the present invention may be any type of electromagnetic radiation including infrared light, ultraviolet light, etc. that may be scattered by a grating or sensor.

For purposes of the present invention, the term “light source” refers to a source of incident light that is scattered by a grating or sensor of the present invention. In one embodiment of the present invention, a light source may be part of a device of the present invention. In one embodiment a light source may be light present in the environment of a sensor or grating of the present invention. For example, in one embodiment of the present invention a light source may be part of a device that is separate from the device that includes the sensors and detector of the present invention. A light source may even be the ambient light of a room in which a grating or sensor of the present invention is located. Examples of a light sources include a laser, a light emitting diodes (LED), an incandescent light bulb, a compact fluorescent light bulb, a fluorescent light bulb, etc.

For purposes of the present invention, the term “line” refers to “line” as this term is commonly used in the field of nanolithography to refer to a one-dimensional shape.

For purposes of the present invention, the term “lipid multilayer” refers to a lipid coating that is thicker than one molecule.

For purposes of the present invention, the term “low humidity atmosphere” refers to an atmosphere having a relative humidity of less than 40%.

For purposes of the present invention, the term “lyotropic” refers to the conventional meaning of the term “lyotropic,” i.e., a material that forms liquid crystal phases because of the addition of a solvent.

For purposes of the present invention, the term “microstructure” refers to a structure having at least one dimension smaller than 1 mm. A nanostructure is one type of microstructure.

For purposes of the present invention, the term “nanostructure” refers to a structure having at least one dimension on the nanoscale, i.e., a dimension between 0.1 and 100 nm.

For purposes of the present invention, the term “plurality” refers to two or more. So an array of microstructures having a “plurality of heights” is an array of microstructures having two or more heights. However, some of the fluorescent microstructures in an array having a plurality of heights may have the same height.

For the purposes of the present invention, the term “reaction chamber” refers a chamber in which one or more sensors of a sensor device are exposed to a fluid present in the chamber. In some embodiments of the present, a reaction chamber may be an open or closed fluid channel through which a sample or other fluids flow. In some embodiments, a reaction chamber may be a chamber into which a sample may be added to expose the one or more sensors to the sample.

For purposes of the present invention, the term “reactive agent” refers to a material or organism bound to, complexed with, inserted in, etc. a lipid multilayer grating that will interact with an analyte of a sample and thereby cause the optical properties of the lipid multilayer grating to change. Examples include functional groups, embedded molecules, embedded ions, membrane bound proteins, living cells, bacteria, nanoparticles, catalysts, etc.

For purposes of the present invention, the term “scattering” and the term “light scattering” refer to the scattering of light by deflection of one or more light rays from a straight path due to the interaction of light with a grating or sensor. One type of interaction of light with a grating or sensor that results in scattering is diffraction.

For purposes of the present invention, the term “sensor” and the term “sensor element” are used interchangeably, unless specified otherwise, and refer to a material that may be used to sense the presence of an analyte.

For purposes of the present invention, the term “square” refers to a microstructure that is square in shape, i.e., has a two-dimensional shape wherein all sides are equal. Although the experiments discussed below in the Example for two-dimensional shapes for microstructures that were squares, the present invention may also be used with other two-dimensional shapes such as rectangles, circles, parallelograms, pentagons, hexagons, etc.

### DESCRIPTION

The interaction of biological cells with the complex and dynamic extracellular environment is a fundamental process that allows the hierarchical organization of life on earth. Direct communication between cells, as well as cellular detection of and influence on non-biological cues is mediated by a variety of chemical and physical signals. For example, in the case of bacterial quorum sensing, recent evidence has demonstrated that, in addition to the concentration of signaling molecules, the local (subcellular) dimensions and confined diffusional properties of the environment influence cellular behavior and the resulting induction of genetic reprogramming. An understanding of, and the ability to control these effects by means of nanostructured environments may enable surfaces to be engineered that may both detect and influence processes such as biofouling, host-pathogen interactions and bioremediation.

The interaction of electromagnetic waves with matter can be controlled by structuring the matter on the scale of the wavelength of light, and various photonic components have been made by structuring materials using top-down or bottom-up approaches. Dip-pen nanolithography is a scanning-probe-based fabrication technique that may be used to deposit materials on surfaces with high resolution and, when carried out in parallel, with high throughput.

In one embodiment, the present invention provides lyotropic optical diffraction gratings composed of biofunctional lipid multilayers with controllable heights between ˜5 and 100 nm that may be fabricated by lipid dip-pen nanolithography. Multiple materials may be simultaneously written into arbitrary patterns on pre-structured surfaces to generate complex structures and devices, allowing nanostructures to be interfaced by combinations of top-down and bottom-up fabrication methods.

In one embodiment, the present invention provides fluid and biocompatible lipid multilayer gratings that allow label-free and specific detection of lipid-protein interactions in solution. This biosensing capability takes advantage of the adhesion properties of the phospholipid superstructures and the changes in the size and shape of the grating elements that take place in response to analyte binding.

Fundamental photonic components can be generated from a large variety of materials by top-down lithography or bottom-up self-assembly. Examples include simple Bragg gratings, stacks and two- or three-dimensional photonic materials. A major challenge lies in the integration of multiple chemical functionalities for the generation of more complex devices, including the readout system, in a simple and efficient way. Top-down microfabrication strives to fabricate smaller structures from a single material, whereas the bottom-up approach seeks to assemble and integrate small components into larger and more complex devices. Dip-pen nanolithography (DPN) is a unique method of micro- and nanofabrication, as it is a direct-write method that allows the bottom-up integration of a variety of materials (especially organic and biological molecules) with both high resolution and high throughput, see Ginger, D, S., Zhang, H. & Mirkin, C. A. The evolution of dip-pen nanolithography, Angew. Chem., Int. Ed. 43, 30-45 (2004) and Salaita, K., Wang, Y. H. & Mirkin, C. A. Applications of dip-pen nanolithography, Nature Nanotech. 2, 145-155 (2007), the entire contents and disclosures of which are incorporated herein by reference.

Phospholipids are fundamental structural and functional components of biological membranes that are both fluid and responsive to external stimuli. Phospholipids in biological systems form the bilayer structure of cellular membranes, as well as a variety of multilayer structures. Examples of lipid multilayers in biological systems include multilamellar cristae in the mitochondria, thylakoid grana and the cisternae of the Golgi apparatus and endoplasmic reticulum. Synthetic phospholipid multilayers can be fabricated by spin-coating, see Mathieu M., Schunk D., Franzka S., Mayer C. and Hartmann N. 2010 J. Vac. Sci. Technol. A 28 953; Mennicke U. and Salditt T. 2002 Langmuir 18 8172; controlling hydration between glass slides, see Trapp M., Gutberlet T., Juranyi F., Unruh T., Deme B., Tehei M. and Peters J. 2010 J. Chem. Phys. 133 164505 Eggeling C. et al 2009 Nature 457 1159; Langmuir-Blodgett deposition, see Pompeo G., Girasole M., Cricenti A., Cattaruzza F., Flamini A., Prosperi T., Generosi J. and Castellano A. C. 2005 Biomembranes 1712 29; laser writing, see Scheres L., Klingebiel B., ter Maat J., Giesbers M., de Jong H., Hartmann N. and Zuilhof H.2010 Small 6 1918; dewetting, see Le Berre M., Chen Y. and Baigl D. 2009 Langmuir 25 2554; Diguet A., Le Berre M., Chen Y. and Baigl D. 2009 Small 5 1661; and dip-pen nanolithography (DPN), see Lenhert S, Sun P., Wang Y. H., Fuchs H. and Mirkin C. A. 2007 Small 3 71, and the entire contents and disclosures of the above articles are incorporated herein by reference.

In the presence of water, phospholipids spontaneously self-organize to form liposomes (or vesicles), which are widely used for a variety of biological and nanotechnological applications. For example, the physical chemistry of liposome adhesion on surfaces is well studied as a model system for cell-surface interactions and surface biofunctionalization in general. Furthermore, liposomes have been used as nanoscale containers with attoliter to zeptoliter volumes and networks for nanoscale transport of materials between vessels. The loading of vesicles (for example, by surface binding, encapsulation or intercalation) with a variety of biofunctional materials such as drugs, nucleic acids and proteins is developed for applications in delivery to biological cells. The structuring of adherent phospholipid multilayers into arbitrary photonic structures according to one embodiment of the present invention therefore provides a new approach for the fabrication and observation of biomimetic nanosystems.

DPN has emerged as a reliable method for creating microstructures with a wide variety of materials on desired surfaces, see Lenhert S. et al 2010 Nat. Nanotechnol. 5 275; Braunschweig A. B., Huo F. W. and Mirkin C. A. 2009 Nat. Chem. 1 353; Lenhert S., Fuchs H. and Mirkin C. A. 2009 Materials Integration by Dip-pen Nanolithography (Weinheim: Wiley-VCH); Zhang H., Amro N., Disawal S., Elghanian R., Shile R. and Fragala J. 2007 Small 3 81; Li B., Goh C F, Zhou X. Z., Lu G., Tantang H., Chen Y. H., Xue C., Boey F. Y. C. and Zhang H.2008 Adv. Mater. 20 4873; Li H., He Q. Y., Wang X. H., Lu G., Liusman C., Li B., Boey F., Venkatraman S. S, and Zhang H. 2011 Small 7 226; Salaita K., Wang Y. H. and Mirkin C. A. 2007 Nat. Nanotechnol. 2 145; Haaheim J. and Nafday O. N. 2008 Scanning 30 137; and Ginger D. S., Zhang H. and Mirkin C. A. 2004 Angew. Chem. Int. Ed. 43 30, the entire contents and disclosures of which are incorporated herein by reference. Using phospholipids as the ink for DPN allows control of the lipid multilayer stacking (height) and biocompatible material integration on solid surfaces, see Sekula S. et al 2008 Small 4 1785; and Wang Y. H., Giam L. R., Park M., Lenhert S., Fuchs H. and Mirkin C. A. 2008 Small 4 1666, the entire contents and disclosures of which are incorporated herein by reference.

The resulting biomimetic lipid structures may be used in cell-surface models, biochemical sensors, drug screening and delivery vehicles, for analysis of cell-cell interactions, and to elucidate the mechanisms of membrane trafficking. Lipid multilayer structures have been fabricated using both serial and massively parallel DPN modes, allowing throughputs on the scale of cm² min⁻¹. The height of phospholipid structures can be tuned by the tip contact time and controlling the relative humidity of the patterning environment in DPN, see Lenhert S., Sun P., Wang Y. H., Fuchs H. and Mirkin C. A. 2007 Small 3 71, the entire contents and disclosure of which are incorporated herein by reference.

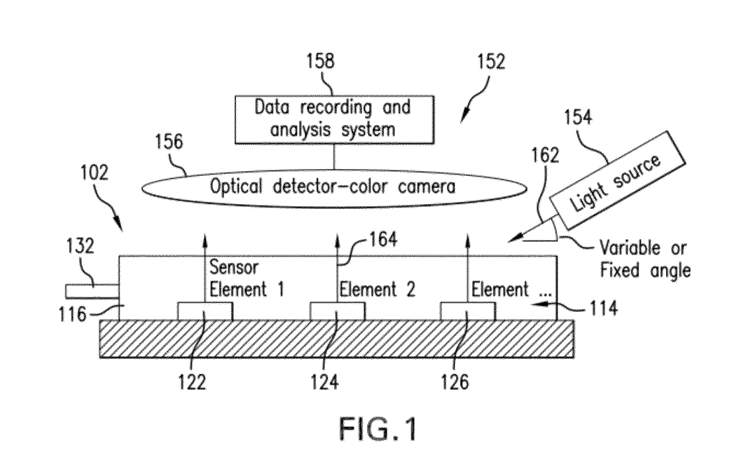

FIG. 1 illustrates a lab-on-a-chip sensor device 102 according to one embodiment of the present invention that made be fabricated using DPN techniques. Sensor device 102 comprises a substrate 112, a sensor array 114 of sensor elements on substrate 112 and a Reaction chamber 116. Although for simplicity of illustration only three sensor elements of sensor array 114 are shown in FIG., 1, i.e., sensor element 122, sensor element 124 and sensor element 126, but sensor array 114 may include additional sensor elements. Each of the sensor elements comprises a lipid multilayer grating. Reaction chamber 116 may be supplied with fluid by a fluid inlet 132. Also shown in FIG. 1 is a detection apparatus 152 comprising a light source 154 and a camera 156. Camera 156 is connected to a data recording and analysis system 158. When illuminated by light source 154 as shown by arrow 162, the sensor elements of sensory array 114 emit light 164, as shown by arrows 164, due to fluorescence.

In one embodiment of the present invention, samples containing one or more analytes may be analyzed in the following manner. Light source 154 is used to illuminate the sensor elements of sensor array 114 so that camera 156 can detect how light is scattered by each of the sensor elements of sensor array 114. Reaction chamber 116 is then supplied with a liquid sample containing analytes through fluid inlet 132. After analytes in the sample are allowed to bind, react, form a complex, etc. with the sensor elements of sensory array 114, light source 154 is used to illuminate the sensor elements and camera 156 is used to detect the light scattered by sensor elements of sensor array 114 after the possible interaction of one or more of the analytes with one or more of the sensor elements. Data recording and analysis system 158 is then used to determine if there has been a change in the optical properties in one or more of the sensor elements of sensor array 114.

The light source used to illuminate the sensor array of FIG. 1 may be provided at a variable or at a fixed angle.

The data recording and analysis system may be part of the camera or may be a separate computer, laptop computer, tablet computer, smartphone, electronic device, electronic instrument, etc. that is in wired or wireless communication with the camera.

Although not shown in FIG. 1, a reaction chamber of the present invention may include an outlet to allow fluid, such as a sample, to flow through the reaction chamber.

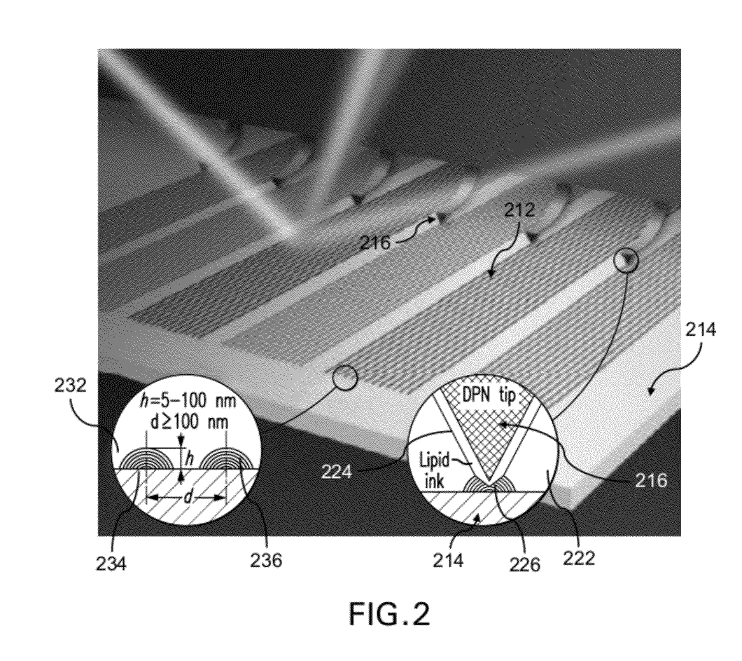

The sensors of the present invention may be made up of various lipids using DPN techniques. For example, fluid phospholipids such as 1,2-dioleoyl-sn-glycero-3-phosphocholine (DOPC) are particularly well suited as biocompatible inks for DPN because of their viscosity, and corresponding properties of ink transport between the DPN tip and substrate, may be readily tuned by the relative humidity, Lenhert, S., Sun, P., Wang, Y. H., Fuchs, H. & Mirkin, C. A. Massively parallel dip-pen nanolithography of heterogeneous supported phospholipid multilayer patterns. Small 3, 71-75 (2007), the entire contents and disclosure of which are incorporated herein by reference. Fluid phospholipids may therefore be used to write on a variety of solid substrates, both smooth surfaced and pre-patterned, without a specific chemical driving force or covalent binding to the surface. Because fluid phosopholipids are biological molecules, a variety of functional membrane lipids (both natural and synthetic) are readily available and can be directly dispersed in the ink for the fabrication of biofunctional structures. These different biofunctions may then be simultaneously written onto the same substrate using different tips in a parallel array, for example, by microfluidic inkwells or inkjet printing, a method referred to as multiplexed DPN, see Wang, Y. H. et al. A self-correcting inking strategy for cantilever arrays addressed by an inkjet printer and used for dip-pen nanolithography. Small 4, 1666-1670 (2008), and Sekula, S. et al. Multiplexed lipid dip-pen nanolithography on subcellular scales for the templating of functional proteins and cell culture. Small 4, 1785-1793 (2008), the entire contents and disclosures of which are incorporated herein by reference. Importantly, the self-organization properties of phospholipids enable them to stack controllably into multilayer structures that are used for optical scattering, as shown schematically in FIG. 2. FIG. 2 shows lipid multilayer gratings 212 deposited on a substrate 214 using DPN tips 216. An inset 222 shows a DPN tip 216 and lipid ink 224 being deposited as a line 226 of one of gratings 212. Inset 232 shows two lines, i.e., lines 234 and 236, of one of arrays 212.

As shown in FIG. 2, parallel DPN tip arrays are used to deposit multiple lipids simultaneously with controllable multilayer heights, laterally structured to form arbitrary patterns such as diffraction gratings with feature sizes on the same scale as UV, visible or infrared light. In situ observation of the light diffracted from the patterns may be carried out during DPN and used for high-throughput optical quality control without the need for fluorescence labels.

The ability of lipid DPN to control the lipid multilayer height constructively is important to forming multilayer structures. With the exception of capillary assembly, the majority of lipid patterning methods are limited to single monolayers or surface-supported lipid bilayers.

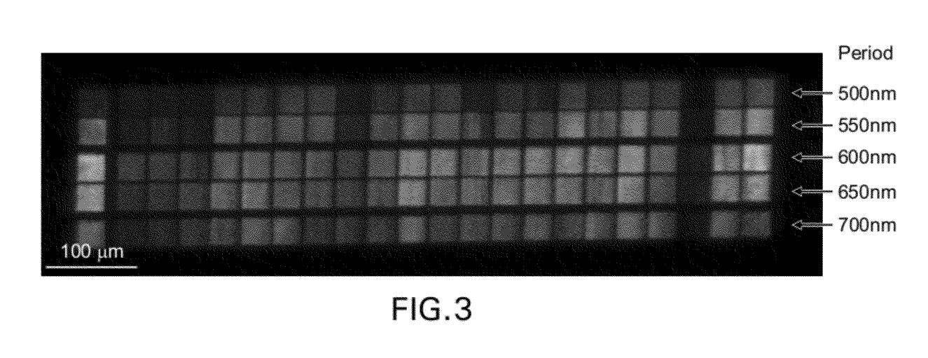

The quality of the structure can be rapidly characterized by illumination of the patterns in a way that allows observation of the light scattering from gratings over large areas, which may also be carried out in situ during DPN fabrication, allowing rapid prototyping of photonic structures. For example, in FIG. 3, the period of the gratings is varied between 500 and 700 nm, and the diffracted light of different colors was detected by a simple charge-coupled device (CCD) camera. Although not visible in the FIG. 3, the gratings having a period of 500 nm are blue in color, the gratings having a period of 550 nm are blue-green in color, the gratings having a period of 600 nm are yellow in color, the gratings having a period of 650 nm are orange in color and the gratings having a period of 700 nm are red in color. FIG. 3 is an optical micrograph of light diffracted from gratings of different period that were fabricated in parallel with a one-dimensional tip array on a poly(methyl methacrylate) (PMMA) surface. Each tip wrote five gratings with periods ranging from 500 to 700 nm in steps of 50 nm (top to bottom).

The different colors observed in FIG. 3 (shown in gray-scale) as a function of grating pitch are described by the grating equation d(sin θₘ+sin θᵢ)=mλ, where d is the period of the grating, θₘ and θᵢ are the angles of diffraction maxima and incidence, respectively, m is the diffraction order, and λ is the wavelength of light. Using white light as incident light, the intensity of light is observed at θₘ≈0° normal to the grating plane. The color observed depends only on the grating period and θᵢ, which is adjusted to give optimal contrast with a period of 600 nm illuminated at θᵢ≈70°.

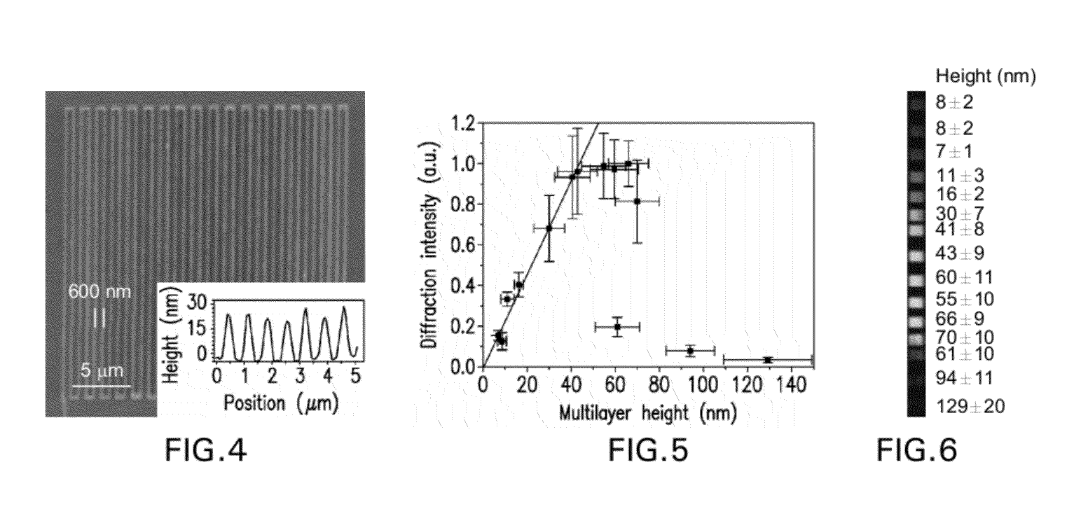

Correlating the grating topographies measured by atomic force microscopy (AFM) with the intensity of light diffracted from the gratings into the camera permits calibration of the observed diffraction intensities as shown in FIGS. 4 and 5. FIG. 4 is an AFM topographical image of a grating with a period of 600 nm and height of (29+3) nm. FIG. 5 is a graph showing the correlation between the grating heights (measured by AFM) and the measured intensity of light diffracted from gratings with a period of 600 nm. The grating efficiency steadily increases linearly to heights of (50+10) nm, after which the multilayer patterns fuse to form a continuous film that no longer diffracts light. A line is fit to the linear region of the data and can be used for optical calibration of the heights. Error bars for the height measurement represent the standard deviation in heights between different grating lines. FIG. 6 is an optical micrograph of the diffraction from the gratings in FIG. 5 and their measured AFM heights. FIG. 5 shows that, for gratings with a period of 600 nm and ink composed of the pure DOPC, the intensity of diffracted light increases linearly with grating height up to 40-60 nm then discontinuously drops off for thicker gratings, because beyond that height, the grating lines fuse together to form a continuous film. The variation in height along a single line is ˜10% of the grating height.

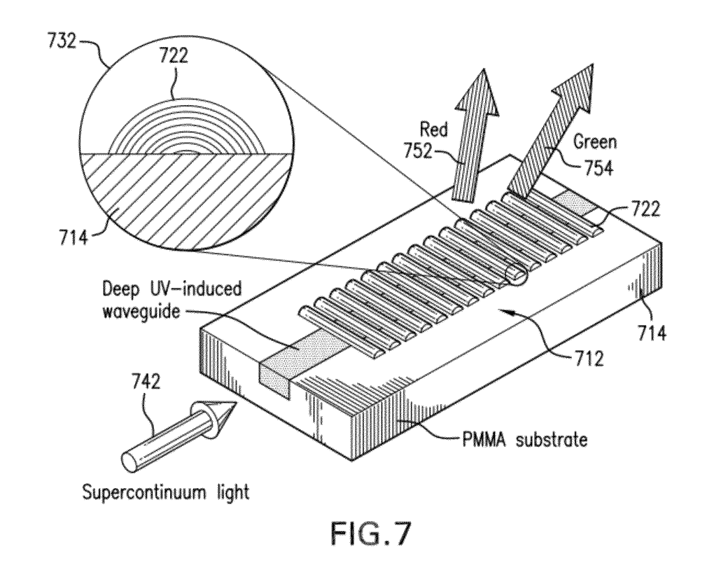

The constructive and parallel nature of DPN makes this technique capable of integrating multiple materials on surfaces that have been pre-structured by top-down lithographic methods for complex device fabrication. As an example, functional waveguide grating coupler as described in Tamir, T. & Peng, S. T. Analysis and design of grating couplers. Appl. Phys. 14, 235-254 (1977), the entire contents and disclosure of which are incorporated herein by reference may be fabricated by direct DPN patterning of DOPC multilayer gratings onto waveguides as shown in FIG. 7. In FIG. 7, the light of a supercontinuum laser source is coupled into a single-mode strip waveguide and decoupled by the waveguide grating coupler, which is a grating based device that couples light in and/or out of a waveguide.

FIG. 7 shows a lipid multilayer grating 712 on a substrate 714. Lipid multilayer grating 712 acts as a grating coupler. Lipid multilayer grating 712 is comprised of lines 722, one of which is shown in greater detail in inset 732. As shown in FIG. 7, light 742 of a supercontinuum laser source (not shown) is couple into a single-mode strip waveguide 744 and decoupled by grating 712. Red light 752 and green light 754 are shown being scattered by grating 712.

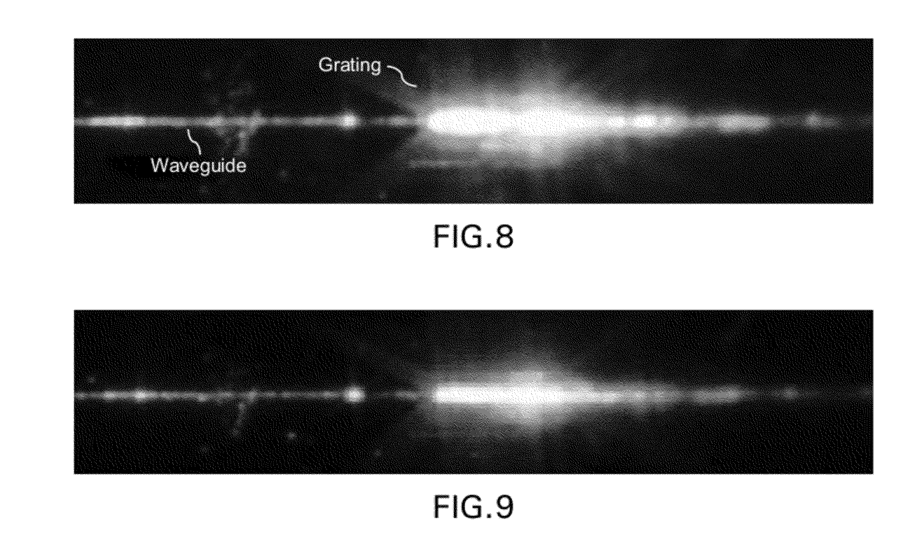

According to one embodiment of the present invention, waveguides have been formed on PMMA surfaces by exposure to deep ultraviolet light through a chromium mask, as described in Henzi, P., Rabus, D. G., Bade, K., Wallrabe, U. & Mohr, J. Low cost single mode waveguide fabrication allowing passive fiber coupling using LIGA and UV flood exposure. Proc. SPIE 5454, 64-74 (2004), the entire contents and disclosure of which are incorporated herein by reference. A lipid grating with a period of 700 nm was defined on top of the UV-induced PMMA waveguide with the lines perpendicular to the waveguide. Light from a supercontinuum laser source (Koheras SuperK Versa) with a spectral emission range of λ=500-800 nm was coupled into the waveguide through an optical fiber. Supercontinuum laser light of different wavelengths was decoupled under different angles by the grating coupler, as shown in FIGS. 8 and 9. FIGS. 8 and 9 are photographs of the coupler at 30° and 45°, respectively, from the surface normal, where the red and green portions of the guided supercontinuum light are coupled to radiation modes. Although not visible in FIGS. 8 and 9, the grating of FIG. 8 is green in color and the grating of FIG. 9 is red color.

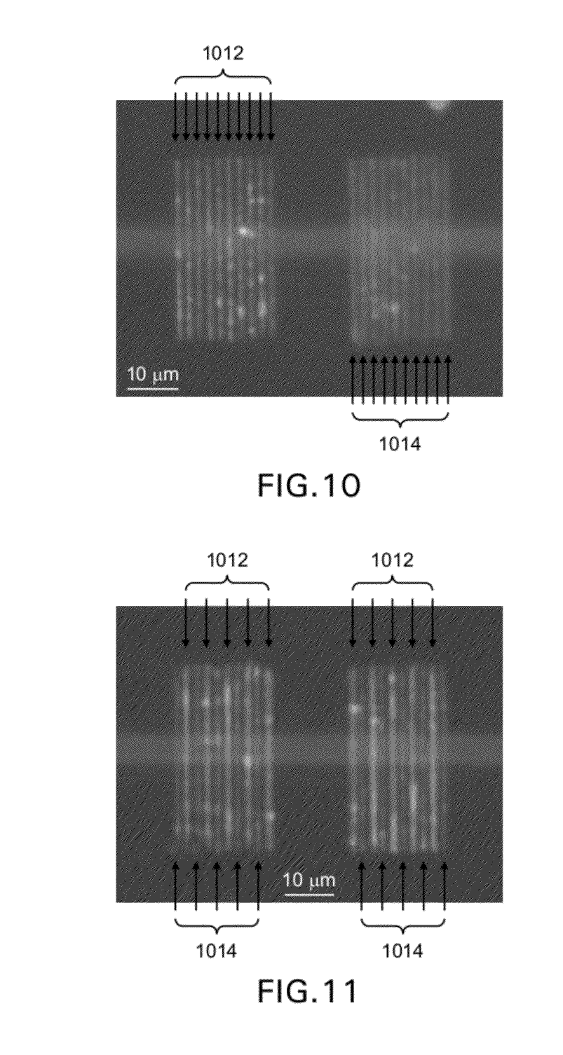

Advanced photonics applications demand the integration of multiple functional materials on micro- and nanoscopic scales and in arbitrary geometries. To demonstrate the qualitatively unique ability of DPN to address this challenge, multiplexed DPN as described in Sekula, S. et al. Multiplexed lipid dip-pen nanolithography on subcellular scales for the templating of functional proteins and cell culture. Small 4, 1785-1793 (2008), the entire contents and disclosure of which are incorporated herein by reference, was used to write two different fluorescently labeled lipids simultaneously on pre-structured waveguides. FIGS. 10 and 11 show a fluorescence overlay of red and green fluorescence from two different fluorescently labeled lipids (red and green vertical lines) integrated with a pitch of 2 μm by DPN patterning on a waveguide (a horizontal thick gray line is visible in FIGS. 10 and 11 because of autofluorescence). In particular, FIG. 10 shows two different gratings simultaneously fabricated from two different tips in a parallel array dipped in inks doped with 1 mol % of fluorescently labeled lipids—rhodamine (red), indicated by arrows 1012, and fluorescein (green), indicated by arrows 1014. FIG. 11 shows two more gratings made with the same tip array and inks, where the individual elements within a single grating are composed of alternating materials. This capability of DPN to control the placement of different materials selectively within a structure opens new possibilities for the rapid prototyping and fabrication of multicomponent photonic structures.

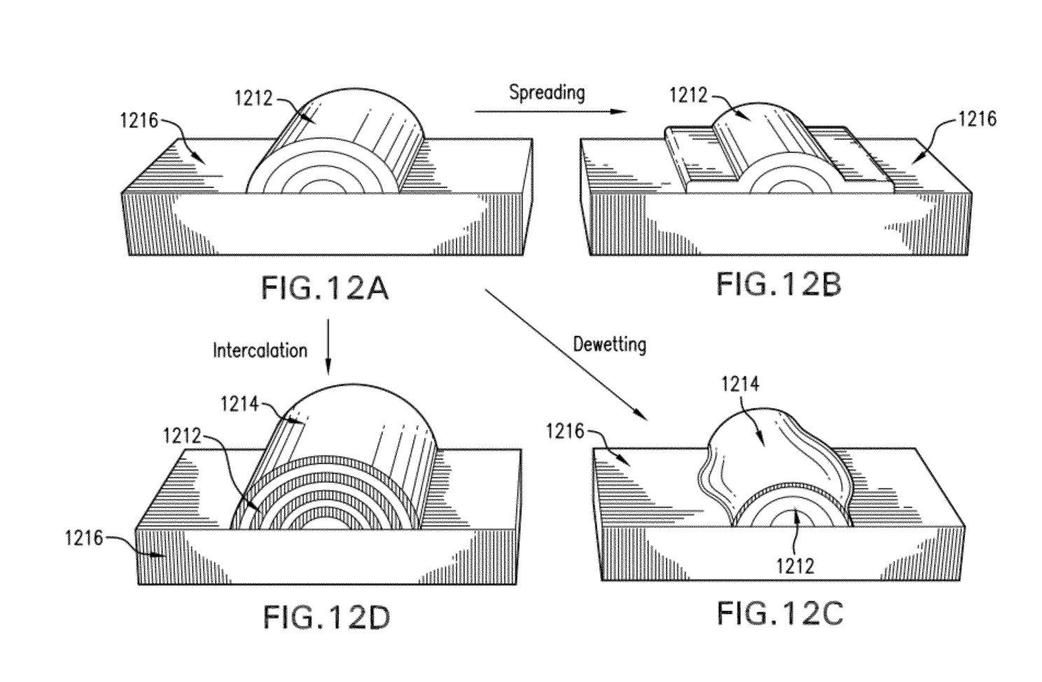

The structuring of lipids into photonic structures provides a label-free method of observing dynamic structural changes in the lipid multilayer morphologies. These changes may be understood in terms of liquid adhesion to a solid surface where the lipid multilayers are, essentially, structured microscopic and nanoscopic oil droplets adherent on a surface. Three examples of shape changes that are spreading, dewetting and intercalation of materials into the multilayer structure, as schematically illustrated in FIG. 12. In FIG. 12, lipid layers are indicated by reference number 1212, protein layers by reference number 1214, and a substrate by reference number 1216. FIG. 12A shows a lipid layers 1212 deposited as a multilayer on substrate 1216. FIG. 12B shows spreading of lipid layers 1212 on substrate 1214. FIG. 12C shows dewetting of lipid layers 1212 with a covering of a protein layer 1214. FIG. 12D shows intercalation of protein layers 1214 with lipid layers 1212.

The drawings A, B, C and D of FIG. 12 have been sketched to reflect the well-documented tendency for hydrated phospholipid multilayers to stack on surfaces into ordered multilamellar bilayer stacks and for hydrophilic materials, such as proteins, to intercalate themselves between the hydrophobic multilayer sheets.

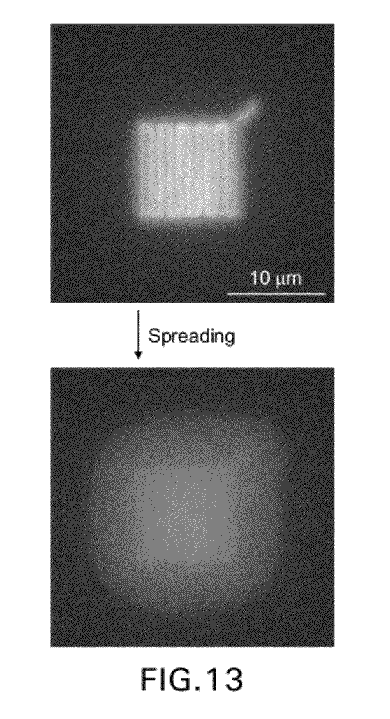

When patterned on surfaces, lipid multilayers are known to spread spontaneously in aqueous solution to form lipid bilayer or monolayer precursor films on certain substrates; see Lenhert, S., Sun, P., Wang, Y. H., Fuchs, H. & Mirkin, C. A. Massively parallel dip-pen nanolithography of heterogeneous supported phospholipid multilayer patterns. Small 3, 71-75 (2007), Sanii, B. & Parikh, A. N. Surface-energy dependent spreading of lipid monolayers and bilayers. Soft Matter 3, 974-977 (2007); 27. Nissen, J., Gritsch, S., Wiegand, G. & Radler, J. O. Wetting of phospholipid membranes on hydrophilic surfaces—concepts towards self-healing membranes. Eur. Phys. J. B 10, 335-344 (1999); Radler, J., Strey, H. & Sackmann, E. Phenomenology and kinetics of lipid bilayer spreading on hydrophilic surfaces. Langmuir 11, 4539-4548 (1995), the entire disclosures and contents of which are incorporated herein by reference. In air, the phospholipid DOPC undergoes a hydration-induced gel-fluid phase transition at a relative humidity of 40%, as observed by humidity-controlled calorimetry and DPN; see Lenhert, S., Sun, P., Wang, Y. H., Fuchs, H. & Mirkin, C. A. Massively parallel dip-pen nanolithography of heterogeneous supported phospholipid multilayer patterns. Small 3, 71-75 (2007), Sanii, B. & Parikh, A. N. Surface-energy dependent spreading of lipid monolayers and bilayers. Soft Matter 3, 974-977 (2007)Ulrich, A. S., Sami, M. & Watts, A. Hydration of DOPC bilayers by differential scanning calorimetry. BBA Biomembranes 1191, 225-230 (1994), the entire contents and disclosures of which are incorporated herein by reference. The multilayer gratings therefore remain stable for long-term storage at low humidity, but upon exposure to humidity higher than 40% in air, the multilayers become hydrated and fluid and therefore slowly spread on the surface. This spreading can be observed both by fluorescence microscopy as shown in FIG. 13 and as a decrease in the diffraction intensity irreversibly indicating the presence of water vapor above 40% humidity. FIG. 13 is a fluorescence micrograph made with fluorescently labeled materials of lipid spreading in air after 5 minutes of exposure to humidity above 40%.

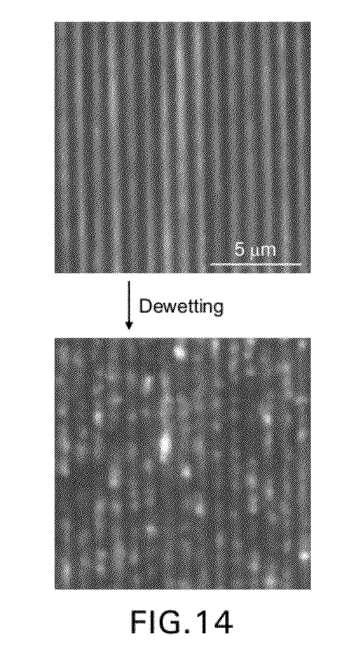

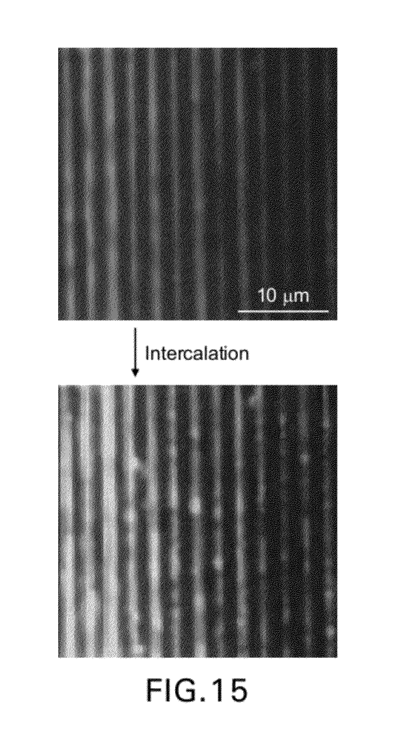

Surprisingly, lipid multilayer gratings can remain stable in aqueous solution for at least several days when immersed under the appropriate conditions, permitting study of the structural changes upon binding of biological molecules such as proteins, which causes the dewetting and intercalation effects observed by fluorescence microscopy and shown in FIGS. 14 and 15. FIG. 14 is a fluorescence micrograph made with fluorescently labeled materials of dewetting of smooth lines of biotin-containing gratings under solution to form droplets after 1 minute of exposure to the protein streptavidin. FIG. 15 is a fluorescence micrograph made with fluorescently labeled materials of intercalation of protein into lipid multilayer grating lines of different heights after 1 hour. The top image is a fluorescence micrograph of fluorescein-(green) labeled lipid grating lines before exposure to protein, and the bottom image shows an image of both red and green fluorescence channels overlaid after binding of a Cy3-(red) labeled protein bound to the layers. Intercalation is indicated because the intensity of fluorescence from bound protein is proportional to the height of the lipid multilayer.

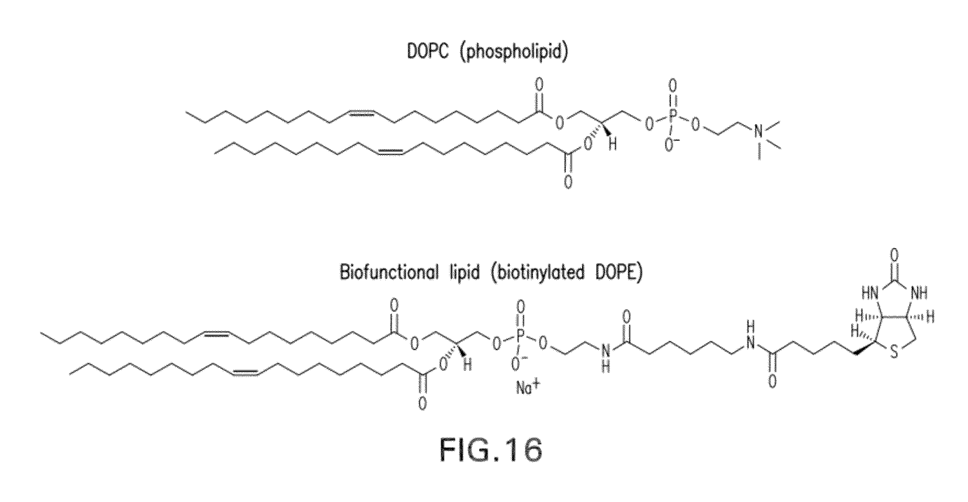

To observe the dewetting and intercalation effects using fluorescence microscopy, DOPC ink was doped with 5 mol % of a biotinylated lipid. The chemical structures of these lipids, i.e., phospholipids (1,2-dioleoyl-sn-glycero-3-phosphocholine (DOPC) and the biotinylated lipid 1,2-dioleoyl-sn-glycero-3-phosphoethanolamine-N-(cap biotinyl)), are shown in FIG. 16. Fluorescently labeled lipids reveal the multilayer grating lines to break into droplets upon exposure to 50 nM of the tetravalent biotin-binding protein streptavidin. Such dewetting or formation of droplets from a continuous line drawn on a surface by a pen is a common practical method of characterizing surface energies by means of dyne pens, see Rentzhog, M. & Fogden, A. Print quality and resistance for water-based flexography on polymer-coated boards: dependence on ink formulation and substrate pretreatment. Prog. Org. Coat. 57, 183-194 (2006), the entire contents and disclosure of which is incorporated herein by reference. In the present case, this method extended to the nanoscale.

Using both fluorescently labeled proteins and lipids, it was possible to observe intercalation of the proteins into lipid multilayers. For example, in the experiment shown in FIG. 1, lines of different multilayer heights were drawn with DOPC containing both a fluorescein-labeled lipid and a biotinylated lipid, as indicated by the different intensities of the fluorescence from the different lines in the green, ‘before’ image of FIG. 16. Upon binding of Cy3-labeled protein (red and green overlay image in FIG. 15), the higher lines can be seen to be significantly brighter than the lower lines, suggesting intercalation into the multilayers after incubation for one hour. Further experiments using fused biotinylated squares of different heights to bind streptavidin, as well as the use of his-tagged green fluorescent protein (GFP) to bind to gratings doped with his-tag binding lipids, confirm the ability of proteins to intercalate themselves within the multilayers.

The tendency for the lipid grating elements to change size and shape upon protein binding, in combination with their optical properties and innate biofunctions, opens the possibility of a new, biologically inspired mechanism for label-free protein detection. Grating-based biosensors are well established and typically function by detecting a spectral change upon analyte binding to the surface of a biofunctionalized solid grating. Although the vast majority of such sensors are made of solid materials, liquid diffraction gratings formed from the directed condensation of water droplets onto chemically patterned surfaces have been proposed as humidity sensors as well as for fundamental studies in adhesion science. The lipid gratings according to one embodiment of the present invention differ from the existing grating based sensors described above in two aspects. First, the biofunctions may be incorporated directly into the phospholipid ink, eliminating the need for further biofunctionalization steps of the transducer as in the case of existing solid gratings. Second, in contrast to the condensation-based liquid gratings, the immiscibility of the adherent liquid phospholipid droplets with water permits studies in biologically relevant aqueous solutions. Procedures for making condensation-based liquid gratings are described in Kumar, A. & Whitesides, G. M. Patterned condensation figures as optical diffraction gratings. Science 263, 60-62 (1994), the entire contents and disclosure of which are incorporated herein by reference.

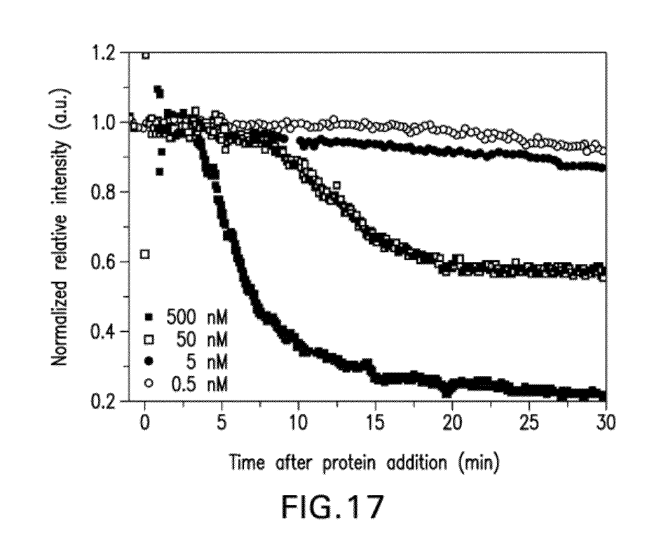

Monitoring the intensity of light diffracted from lipid multilayer gratings on exposure to analytes permits optical detection of protein binding without any fluorescent labels. For example, FIG. 17 shows the optical response of biotinylated gratings upon exposure to streptavidin protein at different concentrations. The decrease in intensity is due to the dewetting mechanism, which results in a lower diffraction efficiency. The observed limit of detection of 5 nM after 15 min is comparable to that of solid grating-based sensors, which are typically diffusion limited at concentrations on the order of ˜5 nM, but after incubation for 90 min, it is possible to observe significant dewetting of biotinylated gratings, as compared to the pure DOPC control gratings, at a protein concentration of 500 pM. As the dewetting detection mechanism depends on a change in surface energy, the sensitivity for a particular analyte may be optimized by adjustment of the sensitivity of the membrane tension to ligand binding, as is the case in many cell-signalling processes and model membrane systems as described in Chiu, D. T. et al. Chemical transformations in individual ultrasmall biomimetic containers. Science 283, 1892-95 (1999), the entire contents and disclosure of which is incorporated herein by reference. Furthermore, phospholipid bilayers are highly resistant to nonspecific protein binding, and it is therefore possible to carry out the same detection of protein added to fetal calf serum. The response of the grating to protein binding depends on the grating height; higher gratings give the best response for protein detection at low concentration. Therefore, observing a quantitative concentration-dependent response requires use gratings of equivalent height (35+5 nm as determined by diffraction intensity calibration) for the experiment series shown in FIG. 17.

Intercalation effects may also be observed by monitoring of diffraction and correspond to increases in the grating volume and therefore height. For example, in the case of streptavidin binding, dewetting and intercalation are observed simultaneously for higher gratings at higher concentrations (for example, 500 nM and above), whereas only intercalation is observed for the lower gratings (for example, FIG. 15). At lower streptavidin concentrations, however, no response is observed for lower gratings, and only dewetting is observed for the higher gratings. Another demonstration of intercalation may be observed by diffraction on binding of a his-tagged GFP protein to nickel-chelating lipid gratings, where the diffraction intensity doubled. Upon addition of imidazole, which releases the his-tag-bound protein, the diffraction intensity could be seen to decrease, and the effect was reversible. Although intercalation and reversibility of the fluid grating response to analytes has so far only been observed for higher, millimolar concentrations, where new sensor constructs are hardly needed, the intercalation mechanism demonstrates the possibility of expanding the dynamic range of disposable sensors. Furthermore, the ability to observe analyte intercalation and desorption from lipid multilayers provides a new, label-free method of characterizing loading and release conditions of liposomes for delivery and nanoscale chemistry applications.

In one embodiment, the present invention provides a process for the fabrication of photonic structures composed of phospholipid multilayers. In one embodiment, the fabrication process of the invention allows direct writing of arbitrary patterns, composed of multiple biocompatible membrane-based materials, on a variety of surfaces, including pre-patterned substrates. The technique is useful for high-throughput biophysical analysis with lipid-based photonic structures and novel photonic sensing elements capable of label-free biosensing by means of a dynamic shape change upon analyte binding. Higher gratings that respond to analyte binding by a surface-tension change are found to be suitable for detection of analytes at low concentrations, whereas mechanisms based on intercalation of materials into the fluid gratings may expand the dynamic range of sensing as well as provide a new way to probe dynamic biomembrane function. The bottom-up fabrication method and unique biophysical properties of nanostructured lipid multilayers permits the integration of complex and dynamic biophotonic circuits.

In one embodiment, the present invention provides in situ detection and control of bacterial quorum sensing based on optical diffraction from nanostructured lipid multilayer arrays. In another embodiment, the present invention provides the formation of printed cellular microarrays on chips to enable systematic studies of the molecular and geometric mechanisms of intercellular communication in quorum sensing. In yet another embodiment, the present invention provides the development of semi-synthetic cell based sensors capable of environmental monitoring.

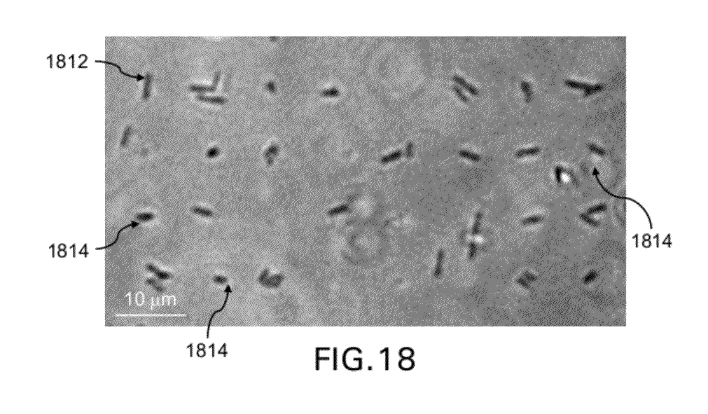

In one embodiment, an array of sensors may have bound to the array a reactive agent such as living bacteria, as shown in FIG. 18.

In one embodiment, the present invention provides a method of monitoring the optical response of lipid multilayer gratings. For this purpose, the gratings may be loaded with different functional groups, antibodies and signaling molecules known to influence quorum sensing and the diffracted light will be quantified as the grating arrays are exposed to different bacterial strains. When a cell attaches to a grating, an optical response is expected, allowing label-free detection of the bacteria. Antibodies will allow detection and selective attachment of target bacterial strains, while the presence of functional groups and signaling molecules in the gratings will allow systematic investigation of how quorum sensing molecules affect cells in a confined environment. FIG. 18 shows how living bacteria may be bound to an array of sensor elements. FIG. 18 is an optical micrograph of E. coli cells 1812 selectively adhered to a fluorescently labeled lipid microarray 1814 (lighter dots). In the experiment shown in FIG. 18, motile Escherichia coli bacteria cells adhere selectively to certain types of phospholipids.

In one embodiment, two or more of lipid multilayer gratings of a device of the present invention may be made from a different lipid or a different mixture of lipids.

In one embodiment of the present invention, the lipid multilayers may serve as models for organic contaminants, as well as model eukaryotic cells, and the bacterial strains will be selected accordingly. Bacterial gene expression may be monitored using cells modified with suitable reporter genes, and mutant strains will be identified by exposure to ionizing radiation and sequencing.

In one embodiment, the present invention provides surface-based arrays composed of different bacterial strains, ideally encapsulated in a lipid matrix in order to allow control of the gene expression of the cells. The bacteria may be purified and mixed with the phospholipid inks prior to DPN printing and deposited in a parallel and multiplexed manner. Cells expressing fluorescent proteins may be used to identify the conditions for printing individual, as well as multiple, cells within each spot. The arrays may then be incubated and the proliferation of the cells monitored by fluorescence measurements in real time. Because DPN allows precise control of spot volume and spacing, the communication between cells in neighboring dots will be studied as a function of dot spacing and size.

In one embodiment, the present invention provides label-free biosensors capable of environmental monitoring. In one embodiment of the present invention, one type of biosensor may be able to identify the different types of bacteria within a population by measuring the specific binding to the arrays. In another embodiment of the present invention, a second type of sensor may contain living bacteria, which will, upon addition of an analyte (e.g. environmental water samples) respond to the presence of materials in the sample as the lipid encapsulated bacteria interact with the sample and the other bacteria on the chip. The chips may then be used to analyze liquid as well as atmospheric samples, as the cells will be trapped within lipid vesicles on the surface. After calibrating these chips with known standards, the structure dependant optical properties of these arrays may provide a versatile and sensitive system based on live yet captive and well organized cells for environmental monitoring.

In one embodiment, the present invention provides a lab-on-a-chip device for mobile multiplexed blood analysis. In another embodiment, the present invention provides a lab-on-a-chip device for screening how different microbes and microbe populations metabolize different oils. Identification of such microbe populations may be useful for environmental monitoring, oil spill cleanup and natural bioremediation.

Although one type of fluorescent dye is described as being used to make the fluorescent lipid multilayer gratings described above, various types of fluorescent additives may be used to make a microstructure a fluorescent microstructure. Examples of suitable fluorescent dyes include various fluorescent organic molecules, fluorescent proteins, pigments, nanoparticles, etc.

The substrate of the present invention may be virtually any type of substrate on which lipid multilayer gratings may be deposited or grown such as glass, plastic, paper, a semiconductor material, etc.

In one embodiment for calibration purposes, the lipid multilayer gratings of the present invention may be fluorescent to allow for the structures of the lipid multilayer gratings to be calibrated using a technique such as described in U.S. patent application Ser. No. 13/234,540, to Lenhert et al. entitled “OPTICAL METHOD FOR MEASURING HEIGHT OF FLUORESCENT PHOSPHOLIPID FEATURES FABRICATED VIA DIP-PEN NANOLITHOGRAPHY” filed Sep. 16, 2011, and the entire disclosure and contents of this application are incorporated herein by reference.

The ability to control the multilayer thickness by the fabrication technique is an important attribute that determines the functionality of lipid multilayers. For example, the efficiency of optical diffraction from lipid multilayer gratings depends on the multilayer thickness, which is a critical factor in their application as model cellular systems and label-free biological sensors, see Tanaka M. and Sackmann E. 2005 Nature 437 656; and Anrather D., Smetazko M, Saba M., Alguel Y. and Schalkhammer T. 2004 J. Nanosci. Nanotechnol. 4 1, the entire contents and disclosures of which are incorporated herein by reference.

### Example

Methods. DPN patterning. A commercial dip-pen nanolithography (DPN) instrument equipped with an environmental chamber (NScriptor, Nanolnk) was used with a one-dimensional tip array with 26 tips (Type F, A26). Optical alignment procedures were used to align the gratings on prefabricated waveguides. Ink preparation (including the use of fluorescently-labeled, biotinylated and metal chelating lipids) was carried out as described elsewhere. The tips were dipped in the inkwells for up to 30 minutes at 23° C. and 70% relative humidity. The gratings were written on PMMA sheets (HESA®Glas HT, Notz Plastics) and treated with isopropanol and ultrapure (Satorius) water (5 minutes sonication both). For total internal reflection fluorescence (TIRF) imaging, PMMA(107 kD, PSS Mainz) was spin-coated from a 15% toluene solution onto glass coverslips to a thickness of 90 nm. The writing process took place at 45% relative humidity and at tip velocities of ranging from 0.1 to 10 μm/s. Substrates before use and the samples after production were stored in a nitrogen atmosphere or vacuum, which minimized hydration-induced spreading.

Grating characterization. Atomic force microscopy measurements were done with a Dimension 3100 (Veeco) in a clean room. Microscopic diffraction images were taken by an inverted TE 2000 fluorescence microscope (Nikon) with a 6× objective and a color camera (Nikon Digital Sight). The gratings were illuminated in transmission through the PMMA substrate by a 150-W halogen cold light source (Schott KL 1500 LCD) at an angle of ca. 70° to the surface normal perpendicular to the grids. Color spectra were measured with a DC 480 microscope (Leica) with a 10× objective connected to a spectrometer (NanoCalc 2000) and a halogen light source (DH-2000 FHS, Mikropack) through four optical fibers in one wire. Fibers 1 and 2 led to the halogen lamp, which illuminated two 40-μm-diameter spots on the surface for orientation purposes; fibers 3 and 4 led to the spectrometers. A 3-mm-wide slot in the optical light path between the objective and the eye piece functioned as a monochromator. TIRF micrographs were taken by the inverted TE 2000 fluorescence microscope (Nikon) with a 100× objective, the W-TIRF illuminator (Nikon), T-PFS perfect focus unit (Nikon), and a CCD camera (Hamamatsu Orca-ER).

Waveguide coupling. A single-mode optical strip waveguide of 8-μm width was obtained by exposure of PMMA to DUV radiation through a quartz-chromium mask. Although the guiding is relatively weak because the low refractive index contrast (ca. 0.005) between that of the exposed surface and the substrate (refractive index of 1.48)3, this method offers a cost-efficient way to fabricate optical waveguides for visible wavelengths. The substrate was 1 mm thick. A mask aligner (EVG 620) operating at a wavelength of λDUV=240-250 nm was used applying an exposure dose of 2 J/cm². A bake at 70° C. for 4 hours after the DUV exposure expelled volatile degradation products and durable radicals from the waveguide.

Protein detection. Protein-sensing experiments were carried out under liquid conditions on an inverted microscope, and time-lapse diffraction images were taken as described above. A homemade rectangular polydimethylsiloxane (PDMS) barrier was placed around the grating and filled with 200 μl of a buffer solution (PBS) containing 0.5% BSA and allowed to incubate for 10 minutes before addition of the streptavidin solution. Unless otherwise noted, the streptavidin solution was added by replacement of half (100 μl) of the solution from the fluid cell with a protein solution of twice the target concentration. Optical noise from sources such as reflection from the fluid cell and air-water interface, solvent evaporation, thermal drift, and general background light was cancelled by division of the signal from the target binding gratings by the signal from pure 1,2-dioleoyl-sn-glycero-3-phosphocholine (DOPC) control grating monitored in parallel on the same surface unless otherwise noted. In order to ensure reproducible immersion of the lipid gratings in solution without induction of structural changes, the solution was added within a glove box in a nitrogen atmosphere, where the lyotropic lipid multilayers are “frozen” by dehydration. Further stability upon immersion was ensured by addition of the 0.5% BSA blocking agent to the solution. BSA prevents spreading of lipid multilayers by binding to the substrate on which the lipid multilayers are deposited. The data were analyzed with ImageJ (v 1.38×) and Origin 6.1 by measurement of the intensity of light measured from a grating and subtraction of the background intensity from it.

Results. The tendency for fluid lipid multilayers to spread under solution, as well as to be disrupted upon crossing the air-water interface, poses a challenge for immersion. It was found that the DOPC-based gratings could be immersed and remained stable under water for at least several days when patterned on hydrophobic PMMA surfaces and when the gratings were immersed in solution containing 0.5% BSA at humidity well below 40% (e.g., in a nitrogen atmosphere). The reason for immersion at low humidity is to freeze the lipids effectively into place so that they are not disrupted by the air-water interface. Further, the 0.5% BSA blocking agent can be expected to block the background surface and slow the spreading. As the difference between spreading behaviors in humid air and under water on hydrophobic surfaces involves different interfacial energies, spreading can be expected to proceed by different mechanisms, i.e., monolayer and bilayer spreading, the kinetics of which has been quantitatively described as a balance between the spreading force and the resistive drag. In contrast to an observable change in contact angle typical for spreading of bulk sessile droplets, lipid multilayers tend to spread as molecularly thin and homogeneous layers. The hydrophobic chain is well known, e.g., from Langmuir-Blodgett films of amphiphilic molecules, to tend to orient toward the air at the air-water interface as well after transfer onto hydrophilic surfaces. Furthermore, evidence has been published that monolayers spread on hydrophobic surfaces under water. The readier spread of lipids may in humid air than under water under the conditions described in this example may be attributed to the difference between these preferred spreading mechanisms and the friction within these molecularly thin precursor films, as well as to surface passivation by the BSA blocking agent.

Having described the many embodiments of the present invention in detail, it will be apparent that modifications and variations are possible without departing from the scope of the invention defined in the appended claims. Furthermore, it should be appreciated that all examples in the present disclosure, while illustrating many embodiments of the invention, are provided as nonlimiting examples and are, therefore, not to be taken as limiting the various aspects so illustrated.

While the present invention has been disclosed with references to certain embodiments, numerous modifications, alterations and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. A device comprising:
a substrate,
one or more sensors on the substrate,
a reaction chamber containing the one or more sensors,
an inlet to the reaction chamber for controlling inflow of fluid into the reaction chamber,
wherein each sensor of the plurality of sensors comprises a lipid multilayer grating.

2. The device of claim 1, wherein device comprises a detector for detecting light scattered by the one or more sensors.

3. The device of claim 2, wherein the device comprises a light source for exposing the one or more sensors to incident light to thereby produce the light that is scattered by the one or more sensors and detected by the detector.

4. The device of claim 1, wherein the one or more sensors comprise a plurality of sensors.

5. The device of claim 4, wherein a first sensor of the plurality of sensors comprises a first lipid and a second sensor of the plurality of sensors comprises a second lipid that is different from the first lipid.

6. The device of claim 4, wherein a first sensor of the plurality of sensors comprises a first reactive agent and a second sensor of the plurality of sensors comprises a second reactive agent that is different from the first reactive agent.

7. The device of claim 1, wherein at least one of the one or more sensors comprises a lipid multilayer grating comprising a mixture of lipids.

8. The device of claim 1, wherein at least one sensor of the one or more sensors comprises a reactive agent.

9. The device of claim 8, wherein the reactive agent is one or more bacteria.

10. The device of claim 8, wherein the reactive agent bound to the lipid multilayer grating of at least one sensor of the one or more sensor elements.

11. The device of claim 8, wherein the reactive agent is complexed with the lipid multilayer grating of at least one of the one or more sensor elements.

12. The device of claim 1, wherein at least one of the sensors of the one or more sensors comprises a dehydrated lipid multilayer grating.
